(19) United States
(12) Patent Application Publication
(10) Pub. No.: US 2012/0264199 A1
(43) Pub. Date: Oct. 18, 2012

(54) PREPARATION METHOD OF THE COMPOUNDS FOR ABSORBING HEAVY METALS

(76) Inventor: Yung-Cheng Lin, Taichung City (TW)

(21) Appl. No.: 13/089,269

(22) Filed: Apr. 18, 2011

(51) Int. Cl. C07K 1/14

(52) U.S. Cl. 435/272

(57) ABSTRACT

The present invention is related to a preparation method of the compounds for absorbing heavy metals. First of all, prepare natural vegetable or fruit and extract a B-M sulfur peptide from the said natural vegetable or fruit. Second, place the natural vegetable or fruit into fermentation and extract the mixed nutrient extraction. And then mix the B-M sulfur peptide and the mixed nutrient extraction as the final extraction. Mix the final extraction into drinks. The final extraction will slowly release B-M sulfur peptide when entering the body; through its combination of heavy metals and re-absorption by the body, useful materials are retained in the human body while long-term accumulated toxic metals are discharged to repair the body's metabolism.

### BACKGROUND OF PRESENT INVENTION

1. Field of Present Invention

The present invention relates to a preparation method of the compounds for absorbing heavy metals.

2. The Prior Arts

Nowadays people live in a polluted environment and are constantly exposed to toxic metals. Due to the development of industrial technology, the amount of toxic heavy metal increases continuously, and we intake a lot of processed foods which contains chemical materials. We eat food which contains pesticides and herbicide; the milk we drink and the meat we eat come from the animals which are regularly injected with antibiotics, steroids and other drugs. We inhale the smoke from vehicles and industrial emissions. The cleaners and skin care products are also marked a wide range of chemical composition, even carcinogenic substances. Home and offices also became the place to preserve toxin: the modern construction materials contain surprisingly methyl ester, toluene, styrene, etc.

Common sources of toxic metals:

Cadmium waste: industrial wastewater, waste gas, cigarette, second-hand smoke, deviate, vehicles, tires, cement, seafood, lime dust, print, ink, etc.;
Nickel: cyanide oil, paint, batteries, etc.;
Aluminum: cable materials, construction materials, cooking appliance, packaging materials, anti-perspirants, antacids, vehicles, etc.;
Tin: toothpaste, pesticide, canned food, dyes, etc.;
Lead: paint, batteries, canned food, ammunition, gasoline, tap-water, cosmetics, hair dyes, solder, pottery glazes, plumbing pipes, etc.;
Mercury: dental fillings. Fish, batteries, paint, etc.;
Arsenic: chemical and glass industry, wood preservatives, the semiconductor industry, optical industry, fungicides, pesticides, etc.;

The source of toxic metals spreads from air pollution, paint, batteries to seafood, drinking water, food, etc. All this explains why many people become extraordinarily tired and even have the diarrhea symptom after shopping, travel or staying in hospital for one day. Among these toxic metals, lead, mercury and arsenic are the former three most toxic ones. These toxins can be absorbed by the body and not easy to be excreted; the toxic metals will accumulate in body fat, bone and other tissues, and then after long-term accumulation cause a wide range of physical and psychological discomfort, including frustration, depression, memory loss, fatigue, joint pain, osteoporosis, autism, dementia, learning disabilities, etc. Unfortunately, these syndromes are not unique, so they are not often associated to the outcome of heavy metal pollution. People ignore the detection of exposure to toxic metal and cannot fund out the real cause of illness, and then delay the timing for treatment.

Here are some examples of illness caused by heavy metals:

Excess lead is associated with fatigue, constipation, insomnia, restlessness, hyperactivity, children's learning disabilities and other issues; excess aluminum is associated with Alzheimer and might result in low phosphorus, which is significant toward bone health. Excess arsenic is associated with fatigue, skin disease, hands and feet tingling and other issues; excess cadmium is associated with fatigue, tissue aging, musculoskeletal pain, anemia and hypertension and other problems. Nevertheless, we might regularly take some medicine; we might drink and smoke occasionally. How can the human body bear these?

One method to eliminate the overdose harmful metals in the human body contains the same idea with phytoremediation. Phytoremediation is now generally considered of a cheaper and convenient method to eliminate the heavy metals in the soil. There are even some scientists who point out that the feature of the plants can be used to mine metallic minerals in the soil. New Jersey successfully regenerated the land from lead pollution due to battery-manufacture. Understanding the plants in the survival strategies under heavy metal helps to manufacture the plants with ability to absorb massive heavy metal by biotechnology.

Basically, a kind of plant which can effectively remove heavy metal pollute should possess the following characteristics: fast growth, the root which can be deeply rooted in the soil, being easy to harvest, the ability to tolerate and accumulate different kinds of heavy metal. Current research about phytoremediation is mainly based on the following two strategies: the first strategy is to improve the ability of accumulate heavy metal through forcing the single gene in plants associated with accumulating heavy metal to perform massively. The second strategy is to embed a set of external genes involved in heavy metal metabolism, absorption and accumulation pathway into the plants by transgenic technology. There are already a lot of successful examples of manufacturing the heavy-metal-resistant plant by transgenic technology. For instance, researchers in Israel embedded the transporter into tobacco, enabling tobacco to live in the environment with high concentration of nickel. Moreover, researchers in Spain embedded the gene which, induced by cadmium, was able to affect the concentration of glutathione in plants into Arabidopsis, discovering that the transgenic plants can live in the environment of high cadmium concentration and accumulate cadmium in the leaves. Similarly, researchers in Japan and New Zealand embedded some genes induced by aluminum into plants and found out that the transgenic technology can indeed enable the transgenic plants to live in the soil with high aluminum concentration.

In view of these researches, a series of researches especially focusing on human's heavy metal excretion mechanism was done. After continuous experiments and many failures, the inventor finally found out a kind of sulfur-rich peptide small molecule whose amount in plants are positively correlative with the toxic level of cadmium, copper, mercury, lead, zinc, etc. The kind of sulfur-rich peptide small molecule is called “B-M sulfur peptide (Biological-Metal Sulfur peptide)”.

### SUMMARY OF PRESENT INVENTION

To solve the problems of the accumulation of heavy metals and the injury it causes in the body, the inventor designed a preparation method of the compounds for absorbing heavy metals.

One target of present invention is to provide a preparation method of the compounds for absorbing heavy metals. In order to achieve the target mentioned above, present invention “a preparation method of the compounds for absorbing heavy metals” comprises 4 steps, which are:

preparing natural vegetable or fruit;

extracting B-M sulfur peptide from the prepared vegetable or fruit;

extracting mixed nutrient extraction by placing the vegetable or fruit in a fermenter;

mixing B-M sulfur peptide and the nutrient complex as a final extraction.

Because the sulfur-rich peptide small molecule synthesized by plants can transport together with heavy metals in the cytoplasm, the benefit heavy metals are sent back to the cell while the harmful or useless heavy metals are excreted. In the environment with heavy metals, plant or yeast will manufacture “BM sulfur peptide”, which increases with the addition of heavy metals;

1. When heavy metal invades a cell, the outer layer of the cell might absorb the heavy metal as the first line of defense.

2. If heavy metal has invaded into a cell, it will destroy most intracellular enzymes and block cell growth. At this point, B-M sulfur peptide is induced by plant cells to be combined with invading heavy metal.

3. Combined with heavy metal, B-M sulfur peptide forms a low molecular weight complex, and quickly eliminates heavy metal to avoid cell injury.

When this extract enter the body, it will slowly release the B-M sulfur peptide, through its combination of heavy metals and re-absorption by the body as mentioned above, keeping the useful material and discharging toxic metals accumulated long in the body to repair the metabolism.

### DETAILED DESCRIPTION OF THE PREFERRED EMBODIMENT

FIG. 1 shows the flow chart of the preferred embodiment of the present invention. As shown in FIG. 1, the steps of present invention are:

(100) provide natural vegetable or fruit:

In the above step, the natural vegetable or fruit refers to the types of edible plants often seen in the human life; following are some examples in this section:

1. Product organs of vegetable plant includes root, stem, leaf, flower, fruit; by product organ category, vegetable are divided into five groups:

(1) Root crop, whose product organ is succulent root or tuberous root; succulent root, for example: radish, carrots, turnip (root-used chadlock), rutabaga and beetroot; tuberous root, for example: yam-bean.

(2) Stem vegetables, whose product organ is stem or modified stem; rhizome, for example: potato, Jerusalem artichoke, lotus root, ginger, water chestnuts, arrowhead, and taro, etc.; terrestrial stem, such as wild rice, asparagus, bamboo shoots, lettuce shoots, kohlrabi, tsatsai and so on.

(3) Leafy vegetables, whose product organ is leaves, leaf strobilus, cluster of leaves or modified leaves. Common leafy vegetables, such as brassica chinensis, mustard, spinach, celery, and amaranth, etc.; leaf strobilus, such as cabbage head, Chinese cabbage, lettuce, and cabbage mustard, etc.; spicy leafy vegetables, such as green onions, chives, coriander and fennel, etc.; bulb vegetables, such as onions, garlic and lily.

(4) Flower vegetables, whose product organ is flowers, fat floral apex or curd, such as broccoli, day lily flowers, spouting broccoli, seaweed vines, artichokes and kale.

(5) Fruit vegetables, whose product organ is matured fruit; eggplant fruit, such as eggplant, tomatoes and peppers, etc.; seedpod, such as beans: soybeans, peas, lentils and winged bean; pepo, such as cucumber, pumpkin, melon, sponge gourd, and snake gourd as well as fresh eaten pepo like watermelon and muskmelon.

2. There are many different types of fruit; fruit can be divided into five types by the structure and the feature:

(1) Berries, whose exocarp is a layer of epidermis, and whose mesocarp and endocarp are almost all pulp quality, such as grapes, tomatoes and so on.

(2) Pepo: the peel forms a hard shell when matured; the endocarp is pulp quality; such as watermelon, melon and so on.

(3) Orange fruits: epidermis forms carpels, such as orange, pomelo, and so on.

(4) Stone fruits: epidermis forms a hard shell with a seed; such as peach, plum and so on.

(5) Pome fruits: the thalamus develops and forms blubber fresh, which coats the ovary; epidermis and mesocarp are next to the fresh; the endocarp forms the core with the seed inside; such as apple, pears and so on.

(101) Extract from the natural vegetable or fruit→B-M sulfur peptide

The B-M sulfur peptide mentioned in the above step is biological metal sulfur peptide, which can be combined with plural divalent ions; that is, the B-M sulfur peptide is combined with heavy metal and forms a low molecular weight complex.

(102) Place the natural vegetable or fruit in a fermentation to extract nutrient complex.

In the above step, in order to maintain the enzyme activity and control the stability, different kinds of vegetable and fruit are stored separately in fermentation in use of biochemistry with different environments by each chemical property of the vegetable and fruit.

The nutrient complex is selected from one of the mineral groups consisting of zinc, silica, biotin, boron, coenzyme, calcium, selenium, folic acid, magnesium, molybdenum, sulfur acid, zinc, iodine, potassium, phosphorus, copper, iron or manganese cluster, or the nutrient complex is selected from one of the vitamin groups consisting of vitamin A, B1, B2, B3, B5, B6, B12, C, D, or E, or the nutrient complex is selected from one of the eupeptic nutrient groups consisting of lipase, papain, lactase, amylase, protease, cellulase, bromelin, red elm, ginseng root, or licorice root; what should be noticed is that the above examples are only for helping to explain common types and appearances of nutrient complex, not to limit the types of nutrient complex in present invention.

To be noticed, the fermentation is selected from one of the fermentation groups consisting of filling-tower type fermentation, tower type fermentation, vertical type fermentation, Waldhof type fermentation, Acetator and Cavitator type fermentation, cyclone tower type fermentation, cylindro-conical type fermentation, air lift type fermentation, deep jet type fermentation, or rotating dick type fermentation.

The most important function of the fermentation is to multiply the microorganisms as expected in the controlled environment and to gain the product. Because the fermentation is not the technical characteristics of present invention, only a brief explanation is stated here: the fermentation contains a mixer, a stirrer gland and bearing of which, a baffle, an air sparger, and so on.

The purpose of the mixer is to break bubbles into small bubbles, to broaden gas-liquid interfacial area for oxygen transportation, to make the thickness of bubble film thinner, to reduce the spread resistance, and to keep every corner's environment inside the fermentation even.

The stirrer gland and the bearing: the most difficult problem in manufacturing the fermentation is to completely seal the fermentation part where the bearing is installed; complete sealing is the necessary condition to keep the fermentation working in the sterile status for a long time.

The baffle is to prevent the occurrence of swirl in the process of mixing and to increase the ventilation effect.

The air sparger is the equipment to transport air into culture medium of the fermentation. The air sparger is generally used alone or alone with mechanical stir. The air sparger cause great differences in the size of bubbles from the air vent, thus, the design of air sparger affects the result a lot. The air sparger can be divided into 3 types: porous sparger, orifice sparger and nozzle sparger (the sparger with open end or sealed partially).

(103) Mix B-M sulfur peptide and the mixed nutrient extraction as an extraction.

In the above step, the extraction can be added into liquid milk, juice, coffee, cocoa, or water; or the extraction can be mixed with solid or powdered milk, juice, coffee, or cocoa; or the extraction can be in forms of powder, capsules or pills.

Because plants are in the mechanism of tolerating and isolating heavy metal under heavy metal environment, biological metal sulfur peptide plays a significant role. Every biological metal sulfur peptide can be combined with seven divalent ions such as cadmium, chromium, zinc, and so on. Intracellular enzyme will be destroyed when metal ions enter the cell. At this point, biological metal sulfur peptide will be combined with these metal ions and form a low molecular weight metal complex to quickly solving the metal toxicity. Afterwards, the metal complex will be transported into vacuoles for use or to be excreted. Here is the explanation under three kinds of situation: when heavy metal invades the cell, the outer layer may absorb the heavy metal as the first line of defense; if the heavy metal entered the cell, it will destroy most intracellular enzyme and block cell growth. At this point, B-M sulfur peptide is induced by plant cells to be combined with invading heavy metal. Combined with heavy metal, B-M sulfur peptide forms a low molecular weight complex, and quickly eliminates heavy metal to avoid cell injury.

By this way, when this extract enters the body, it will slowly release the B-M sulfur peptide, through its combination of heavy metals and re-absorption by the body, keeping the useful material and discharging toxic metals accumulated long in the body to repair the metabolism mechanism.

Besides, since the nutrient complex used in present invention is extracted from natural vegetable and fruit, it is different from common mineral from chemical synthesis or the so-called natural food which is just mixed food; the nutrient complex can be easier used by the human body than single molecular complex and can stay in the human body for more time.

The above embodiment illustration and examples are only the preferred embodiment in present invention. Those examples above should not, however, be considered to limit the scope of present invention, it is contemplated that modifications will readily occur to those skilled in the art, which modifications will be within the spirit of present invention and the scope of the appended claims.

### BRIEF DESCRIPTION OF THE DRAWINGS

The present invention will be apparent to those skilled in the art by reading the following detailed description of a preferred embodiment thereof, with reference to the attached drawings, in which:

FIG. 1 shows the flow chart of the preferred embodiment in accordance with the present invention, in which 101-103 are the steps of the present invention.

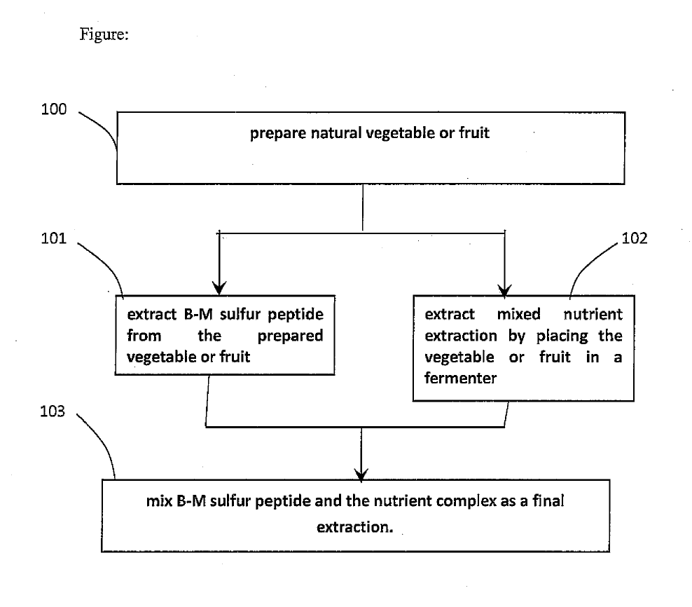

What is claimed is:

1. A preparation method of the compounds for absorbing heavy metals, comprising:
preparing natural vegetable or fruit;
extracting B-M sulfur peptide from the prepared vegetable or fruit;
extracting mixed nutrient extraction by placing the vegetable or fruit in a fermenter; and
mixing B-M sulfur peptide and the nutrient complex as a final extraction.

2. A preparation method of the compounds for absorbing heavy metals as claimed in claim 1, wherein the B-M sulfur peptide is biological metal peptide.

3. A preparation method of the compounds for absorbing heavy metals as claimed in claim 1, wherein the B-M sulfur peptide, combined with heavy metal, forms low molecular weight complex.

4. A preparation method of the compounds for absorbing heavy metals as claimed in claim 3, wherein the B-M sulfur peptide can be combined with plural divalent ions.

5. A preparation method of the compounds for absorbing heavy metals as claimed in claim 1, wherein the final extraction is to be added into liquid milk, juice, coffee, cocoa or water, or to be mixed with solid or powdered milk, juice, coffee, or cocoa, or to be in forms of powder, capsule or pills.

6. A preparation method of the compounds for absorbing heavy metals as claimed in claim 1, wherein the nutrient complex is selected from one of the mineral groups consisting of zinc, silica, biotin, boron, coenzyme, calcium, selenium, folic acid, magnesium, molybdenum, sulfur acid, zinc, iodine, potassium, phosphorus, copper, iron or manganese cluster.

7. A preparation method of the compounds for absorbing heavy metals as claimed in claim 1, wherein the nutrient complex is selected from one of the vitamin groups consisting of vitamin A, B1, B2, B3, B5, B6, B12, C, D, or E.

8. A preparation method of the compounds for absorbing heavy metals as claimed in claim 1, wherein the nutrient complex is selected from one of the eupeptic nutrient groups consisting of lipase, papain, lactase, amylase, protease, cellulase, bromelin, red elm, ginseng root, or licorice root.

9. A preparation method of the compounds for absorbing heavy metals as claimed in claim 1, wherein the fermentation is selected from one of the fermentation groups composed of filling-tower type fermentation, tower type fermentation, vertical type fermentation, Waldhof type fermentation, Acetator and Cavitator type fermentation, cyclone tower type fermentation, cylindro-conical type fermentation, air lift type fermentation, deep jet type fermentation, or rotating dick type fermentation.
